(19) United States
(12) Patent Application Publication
(10) Pub. No.: US 2024/0416129 A1
(43) Pub. Date: Dec. 19, 2024

(54) COGNITIVE FUNCTION IMPROVEMENTS AND BIOMARKER-MODULATED DEEP BRAIN STIMULATION

(71) Applicant: Mayo Foundation for Medical Education and Research, Rochester, MN (US)

(72) Inventors: Michal T. Kucewicz, Rochester, MN (US); Gregory A. Worrell, Rochester, MN (US); Vaclav Kremen, Rochester, MN (US); Victoria S. Marks, Rochester, MN (US); Michal Lech, Gdynia (PL)

(21) Appl. No.: 18/599,917

(22) Filed: Mar. 8, 2024

Related U.S. Application Data
(60) Provisional application No. 63/450,935, filed on Mar. 8, 2023.

(51) Int. Cl. A61N 1/36; A61N 1/05; A61N 1/372; G16H 20/40; G16H 50/30

(52) U.S. Cl. CPC A61N 1/36139; A61N 1/0534; A61N 1/36064; A61N 1/36171; A61N 1/37247; G16H 20/40; G16H 50/30

(57) ABSTRACT

Verbal memory impairment is a common symptom of temporal lobe epilepsy (“TLE”). The ability to encode and recall verbal memories can be probed with the classic free recall task. This study had three overarching hypotheses: 1) increased seizure frequency will lead to decreased verbal memory, 2) stimulation will modulate memory performance, and 3) stimulation will modulate the underlying neural correlates of memory. The study investigated these hypotheses in patients receiving chronic DBS to bilateral ANT. Recordings were obtained from individuals with drug resistant mesial TLE implanted with an investigational MEDTRONIC SUMMIT RC+S™ sensing and stimulation device. As patient seizure diaries are notoriously inaccurate, the patient's continuous local field potential (“cLFP”) was scored for seizures using a validated seizure classifier with trained epileptologist review, resulting in a reliable seizure diary. The subjects completed free recall memory tasks in their home environment with cLFP and behavioral data streamed to a handheld device and cloud repository.

Through implementation of generalized linear mixed models (GLMM), the study was able to determine that ANT stimulation modulates memory performance, but changes in the seizure rate were not predictive of changes in memory performance. Analyzing the continuous local field potential recordings from these tasks, the study found that spectral power in the theta frequency band in the left ANT and hippocampus correlated with acute memory performance and chronic therapeutic stimulation frequency received.

Not all patients are candidates for devices with chronic sensing capabilities, and this study indicates that changes in verbal memory performance can provide a suitable metric of therapy effectiveness in lieu of a reliable, objective seizure diary. But for those who do receive these and similar devices, thalamic-hippocampal spectral activities can be used to track and predict memory performance acutely and chronically, as well as predict the effect of therapeutic deep brain stimulation. Our study demonstrates chronic improvement of verbal memory with a new biomarker-based technology for remote task administration and modulation of the associated neural activities.

### CROSS-REFERENCE TO RELATED APPLICATIONS

This application claims the benefit of U.S. patent application Ser. No. 63/450,935, filed on Mar. 8, 2023. The disclosure of the prior application is considered part of (and is incorporated by reference in) the disclosure of this application.

### STATEMENT OF FEDERALLY SPONSORED RESEARCH

This invention was made with government support under NS095495 awarded by the National Institutes of Health. The government has certain rights in the invention.

### BACKGROUND

### 1. Technical Field

This specification relates to brain stimulation and sensing, including techniques for improving cognitive processes (e.g., memory encoding and recall) using brain stimulation modulated based on theta-band biomarkers from electrical brain activity signals detected in the hippocampus and anterior nuclei of the thalamus.

### 2. Background Discussion

Epilepsy is a common neurological disorder. It has been estimated that the prevalence of epilepsy worldwide is as high as 621.5 per 100,000 people. Approximately one third of people with epilepsy have drug-resistant epilepsy (“DRE”). Many people with DRE are not candidates for epilepsy surgery or continue to have seizures despite surgery, and for these individuals, electrical brain stimulation is a viable treatment option.

Since the clinical success of Deep-Brain Stimulation (“DBS”) therapy for treating motor symptoms in Parkinson's disease, dystonia, and other movement disorders, it has been applied in a range of neurological and neuropsychiatric conditions, including obsessive compulsive disorder, Tourette's syndrome, and depression. New applications are targeting mental disorders to treat more cognitive functions. Declarative memory is one of these functions: explored using DBS and less invasive electrical stimulation methods with inconsistent results. Positive effects reported in one study were challenged in other studies and focused mainly on acute effects on task performance. Clinical trials of DBS for treating memory deficits in neurodegenerative disorders have so far had limited success. Stimulation of the hippocampal fornix failed to restore memory functions in Alzheimer's disease even though it had a positive effect on flashback rates. Another trial of DBS in the nucleus basalis of Meynert for Lewi body dementia also obtained limited effects in single case reports, despite promising results of the preceding studies. Conclusive evidence for a positive DBS effect on declarative memory performance is lacking robust and reproducible results.

A principled approach based on electrophysiological biomarkers of neural activities may be one plausible strategy for DBS studies of cognitive functions.

### SUMMARY

This specification describes systems, methods, devices, and techniques for assessing and improving a cognitive process of a patient through brain stimulation and sensing.

A first aspect includes modulating a brain stimulation therapy to affect a cognitive process of a patient. A data acquisition system acquires electrical brain activity signals sensed by one or more electrodes in a brain of the patient, where the electrical brain activity signals include at least one signal indicative of electrical activity in a hippocampal region of the brain and at least one signal indicative of electrical activity in an anterior nucleus of the thalamus (ANT) region of the brain. A signal analysis system processes the electrical brain activity signals to determine values for one or more features of the electrical brain activity signals. Based on the values for the one or more features of the electrical brain activity signals, a stimulation control system determines one or more parameters for the brain stimulation therapy related to the cognitive process of the patient. Brain stimulation therapy is then administered to the patient in accordance with the determined parameters.

These and other aspects can further include one or more of the following features.

The electrical brain activity signals can include a first signal indicative of electrical activity in a right portion of the ANT region and a second signal indicative of electrical activity in a right portion of the hippocampal region. The one or more features of the electrical brain activity signals can include at least one of a power or a coherence of theta-band electrical activity in the right portion of the ANT region and the right portion of the hippocampal region. The cognitive process includes memory encoding. Theta-band electrical activity can include theta brain waves in the range 2-14 Hz or a narrower band, commonly indicated in the range 4-8 Hz.

The system can acquire electrical brain activity signals sensed by a single electrode in the ANT region or the hippocampal region of the brain, and the features can include a power of the theta-band electrical activity in the ANT or hippocampal region where the single electrode is positioned.

The system can acquire electrical brain activity signals sensed by at least one electrode in the ANT region and at least one electrode in the hippocampal region. The features can include a power of the theta-band electrical activity in the ANT and/or hippocampal regions, a coherence of the theta-band electrical activity, or both.

The electrical brain activity signals can include a first signal indicative of electrical activity in a left portion of the ANT region and a second signal indicative of electrical activity in a left portion of the hippocampal region. The one or more features of the electrical brain activity signals can include at least one of a power or a coherence of theta-band electrical activity in the left portion of the ANT region and the left portion of the hippocampal region. The cognitive process can include memory recall.

The one or more parameters for the brain stimulation therapy can be determined using a model that relates at least one electrical brain activity signal feature to at least one brain stimulation therapy parameter determined to affect the cognitive process. The model can define target values for the one or more features of the electrical brain activity signals, and determining the one or more parameters for the brain stimulation therapy can include comparing the values for the one or more features of the electrical brain activity signals determined from the electrical brain activity signals to the target values for the one or more features.

The one or more parameters for the brain stimulation therapy can include at least one of a stimulation frequency, a stimulation start time, a stimulation end time, or a selection of one or more stimulation sites in the brain of the patient.

The brain stimulation therapy can be DBS.

The patient may be diagnosed with epilepsy and the brain stimulation therapy can be administered at least in part to treat symptoms of epilepsy. More generally, the patient may be diagnosed with a neurological disease that manifests symptoms such as verbal memory deficits, and the brain stimulation therapy can be administered at least in part to treat symptoms such as the verbal memory deficit.

A type or a time of an anticipated cognitive process can be identified, and the one or more parameters for the brain stimulation therapy can be determined based on at least one of the type or the time of the anticipated cognitive process. The type of the anticipated cognitive process can be selected from a group that includes memory encoding and memory recall.

The electrical brain activity signals can include local field potentials (“LFPs”) sensed by the one or more electrodes in the brain of the patient.

A second aspect can include assessing a cognitive process of a patient. A data acquisition system acquires electrical brain activity signals sensed by electrodes in a brain of the patient, where the electrical brain activity signals include at least one signal indicative of electrical activity in a hippocampal region of the brain and at least one signal indicative of electrical activity in the ANT region of the brain. A signal analysis system processes the electrical brain activity signals to determine values for one or more features of the electrical brain activity signals. The signal analysis system determines a cognitive score indicative of a predicted level of cognition of the patient with respect to the cognitive process and provides an output based on the cognitive score.

These and other aspects can further include one or more of the following features.

The electrical brain activity signals can include a first signal indicative of electrical activity in a right portion of the ANT region and a second signal indicative of electrical activity in a right portion of the hippocampal region. The one or more features of the electrical brain activity signals can include at least one of a power or a coherence of theta-band electrical activity in the right portion of the ANT region and the right portion of the hippocampal region. The cognitive process can include memory encoding.

The electrical brain activity signals can include a first signal indicative of electrical activity in a left portion of the ANT region and a second signal indicative of electrical activity in a left portion of the hippocampal region. The one or more features of the electrical brain activity signals can include at least one of a power or a coherence of theta-band electrical activity in the left portion of the ANT region and the left portion of the hippocampal region. The cognitive process can include memory recall.

The output can be provided by recording data indicative of the cognitive score in a memory device, presenting data indicative of the cognitive score on an electronic display, and/or transmitting data indicative of the cognitive score over a communications network.

The cognitive score can be used in a process to determine parameters for a brain stimulation therapy, and the brain stimulation therapy can be administered to the patient in accordance with determined parameters.

The cognitive score can indicate a predicted level of an acute memory encoding ability or an acute memory recall ability of the patient.

The cognitive score can indicate a predicted level of a chronic memory encoding ability or a chronic memory recall ability of the patient.

The cognitive score can be further determined based on an amount of time that the patient has been administered chronic deep brain stimulation, historical values for the one or more features of earlier-acquired electrical brain activity signals, a frequency of deep brain stimulation administered to the patient, an indication of the patient's sleep quality, an indication of the patient's mood, and/or demographic information about the patient.

A third aspect involves adjusting a brain stimulation therapy by performing actions that include: obtaining results of one or more non-invasive cognitive performance assessments administered to the patient, where the results are indicative of at least one of a memory encoding ability or a memory recall ability of the patient; based on the results of the one or more non-invasive cognitive performance assessments, determining one or more parameters for the brain stimulation therapy; and applying the brain stimulation therapy to the patient in accordance with the one or more parameters. The one or more non-invasive cognitive performance assessments can include a free recall task. The one or more parameters for the brain stimulation therapy can correlate with a power of theta-band electrical activity in at least one of a hippocampal region of the brain or an ANT region of the brain. The one or more parameters for the brain stimulation therapy can correlate with a coherence of theta-band electrical activity across the hippocampal region and ANT regions of the brain.

In an aspect, a system comprises circuitry configured to perform any of the methods disclosed herein. The circuitry can include software, hardware, digital electronics, analog electronics, or a combination of these.

In an aspect, one or more non-transitory computer-readable media are encoded with instructions that, when executed by one or more processors, cause the one or more processors to perform the actions, methods, and processes disclosed herein.

Additional features and advantages will be apparent to one of ordinary skill in view of the specification, the figures, and claims.

### BRIEF DESCRIPTION OF DRAWINGS

FIG. 1 is a diagram of an example system for acquiring and analyzing electrical brain activity signals and controlling a brain stimulation therapy to improve a cognitive process of a patient.

FIG. 2 is a flowchart of an example process for adapting a brain stimulation therapy to improve a cognitive process of a patient.

FIG. 3 is a flowchart of an example process for assessing a cognitive process of a patient using theta-band features from the hippocampus and anterior nucleus of the thalamus.

FIG. 4 is a flowchart of another example process for adapting a brain stimulation therapy to improve a cognitive process of a patient.

FIG. 5 illustrates performance of a free recall task for memory assessments.

FIG. 6 is a table of information from an example study that indicates, for each patient, the patient's therapy length, number of seizures, number of memory task sessions, and percentage data streamed.

FIG. 7 depicts a set of plots indicative of dense cognitive and seizure count tracking in the studied patients. Four patients (Subject 1, Subject 2, Subject 3, and Subject 4) participated in sessions of remote, free recall tasks. Session performance is measured in percent remembered (left y-axis) with mean and standard deviation across session lists reported (black line with grey shading). Each session is marked with either a circle or a triangle for whether a patient was receiving low or high frequency stimulation that week. Weekly seizure counts (right y-axis) are represented by the histogram bars.

FIG. 8 is a plot of seizure count over time from patients in the example study. Each patient is represented by a different plot line, with task days marked in either a circle or a triangle, depending on if they were set to low or high frequency stimulation, respectively. The top hashed line indicates the GLMM predicted seizure count during low frequency stimulation, and the lower hashed line indicates the GLMM predicted seizure count during high frequency stimulation.

FIG. 9 is a plot of memory score over time of patients in the example study. The GLMM predicts that memory performance will be better when patients are receiving lower frequency therapeutic stimulation than higher frequency. Each patient is represented by a different plot line with task days marked in either a circle or a triangle, depending on if they were set to low or high frequency stimulation, respectively. The upper hashed line indicates the GLMM predicted memory performance during low frequency stimulation, and the lower hashed line indicates the GLMM predicted memory performance during high frequency stimulation.

FIGS. 10A and 10B provide plots from the example study of theta power and coherence across the anterior thalamic nuclei and the hippocampi. (a) Theta power was significantly induced by the display of subsequently recalled words in the right thalamus and hippocampus (left column). Subsequently forgotten words, on the other hand, induced a significant theta power response in the left thalamus and hippocampus (middle column). Subsequent memory effect was observed in the left thalamus and hippocampus where theta power was significantly lower before the presentation of the forgotten compared to the recalled words. (b) Analogous analysis for coherence revealed significantly induced connectivity between both hippocampi on the recalled word trials (left column) and between the right thalamus and hippocampus on both recalled and forgotten word trials (left and middle columns). The subsequent memory effect was again observed between the left thalamus and hippocampus (LT-LH) with coherence significantly greater before presentation of the recalled words, and between both hippocampi (LH-RH) where the theta coherence was significantly lower before presentation of the recalled words. Both thalamic and hippocampal power and coherence predict subsequent recall in the period immediately preceding word encoding.

FIGS. 11A and 11B depict plots from the example study of population-average alpha power and coherence changes during the word display event for recalled and forgotten words dependent on brain area and recall state. (a) Alpha power was significantly induced by the display of subsequently recalled words in the right hippocampus (left column). Subsequently forgotten words, on the other hand, induced a significant alpha power response in the left thalamus (middle column). Subsequent memory effect was not observed in alpha power. (b) Analogous analysis for coherence revealed no significantly induced alpha connectivity for the recalled word trials (left column) or the forgotten word trials (middle column). The subsequent memory effect was observed between the right thalamus and hippocampus (RT-RH) with coherence significantly greater before presentation of the recalled words.

FIGS. 12A and 12B depict plots from the example study of population-average gamma power and coherence during the word display event for recalled and forgotten words were dependent on brain area and recall state. (a) Gamma power was significantly induced by the display of subsequently forgotten words in the left thalamus (middle column). Subsequent memory effect was not observed in gamma power (right column). (b) Analogous analysis for gamma coherence revealed significantly induced connectivity between no brain areas. The subsequent memory effect was not observed for gamma coherence for any combination of brain areas observed in this study.

FIGS. 13A through 13C depict plots from the example study of longitudinal memory performance and the effect of deep brain stimulation in the thalamic-hippocampal theta activities. (a) Individual patient examples show progressive improvement in memory performance with low-frequency (2 Hz) ANT stimulation mode (violet background) and gradual deterioration in performance with high-frequency (145 Hz) DBS (yellow). Pink indicates 7 Hz DBS, and white background indicates no chronic DBS. (b) Mean memory performance is correlated with the mean theta power at word onset (background DBS color-coded as in (a)). (c) Patients recalled fewer words and showed less theta power at word onset in the left thalamus (Subject 1) and the left hippocampus (Subject 4) during the high-frequency stimulation mode of chronic DBS. A subset of sessions from (a) with robust data was used in (b) and (c).

FIG. 14 depicts components of example computing devices and systems that can be implemented in the computer-based aspects of the techniques disclosed herein.

### DETAILED DESCRIPTION

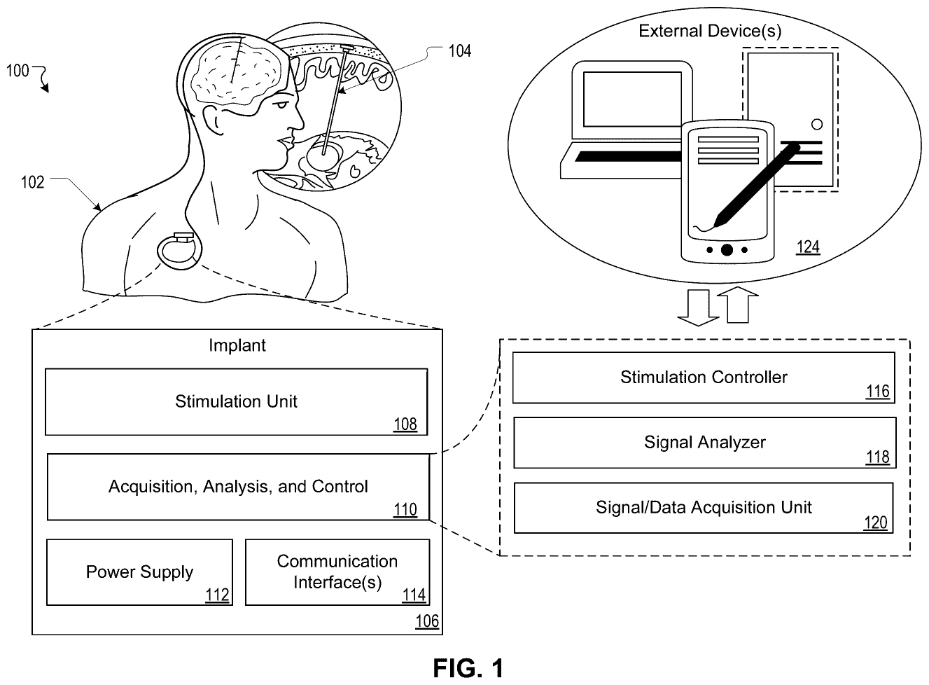

FIG. 1 is a block diagram of an example brain stimulation and sensing system 100, which is especially adapted to facilitate sensing and stimulation directed at least in part to assessing and improving a patient's cognitive functions.

System 100 includes an implantable device 106, e.g., which may be implanted sub-dermally beneath the collarbone of a patient 102. The patient 102 in this example is a human, although the techniques disclosed in this specification may be extended for use with other mammals as well. In some implementations, implantable device 106 is equipped with a stimulation unit 108, a power supply 112 (e.g., a battery), and one or more wired or wireless communication interfaces 114. The implantable device 106 may further include or communicate with acquisition, analysis, and control circuitry 110. The acquisition, analysis, and control circuitry 110 can include a stimulation controller 116, signal analyzer 118, and signal/data acquisition unit 120, each of which may be embedded in the implantable device 106, implemented in external devices 124, or whose functions may be partially split among embedded and external components.

In general, stimulation unit 108 is configured to generate and deliver via wired leads electrical brain stimulation signals to electrodes 104 in the patient's brain. The stimulation and sensing electrodes may each be implanted in one or more regions of the brain, and in some implementations, sensing and stimulation electrodes are both provided in the anterior nuclei of the thalamus (“ANT”) and the hippocampus. Although the sensing electrodes may be distinct from the stimulating electrodes in certain implementations, in other cases the same set of electrodes (or a subset of electrodes) may be used both for stimulation and sensing. The dual use of an electrode for stimulation and sensing can be achieved, for example, through interleaved sampling in which the same electrode is alternately used to deliver one or more stimulation pulses between sensing periods in which the electrode records an iEEG signal. Stimulation unit 108 can be an implantable pulse generator, for example, and can deliver continuous brain stimulation, responsive stimulation, adaptive stimulation, or a combination of these.

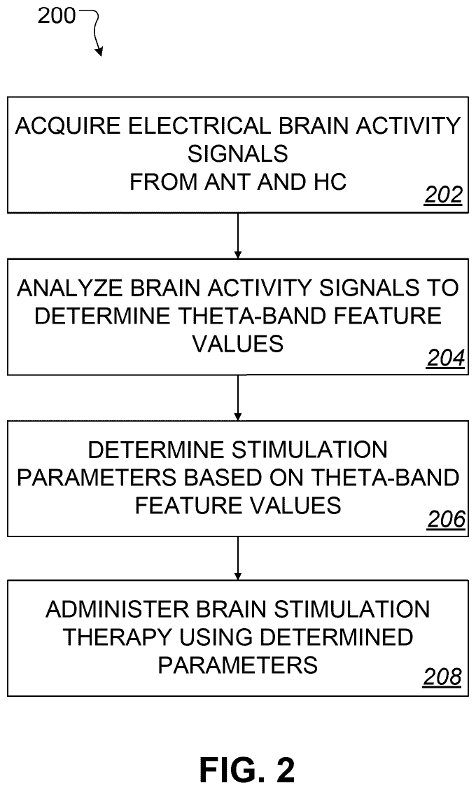

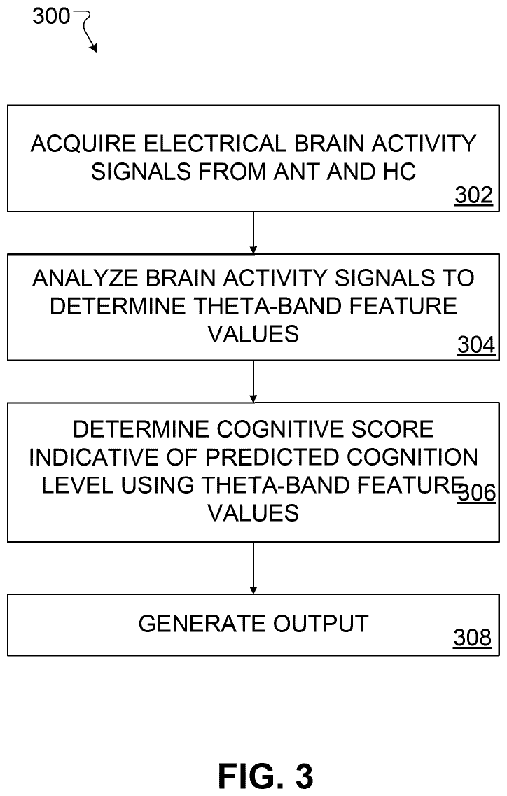

Acquisition, analysis, and control circuitry 110 includes a number of components for acquiring electrical brain activity signals. The signal/data acquisition unit can continuously, regularly, or otherwise at programmed times capture brain activity signals from the sensing electrodes in the patient's brain. Signal acquisition circuitry 114 may include amplifiers, filters, and an analog-to-digital (A/D) converter, for example, to obtain digitized versions of electrical brain activity signals that can be processed with digital processing components of the system. Signal analyzer 118 includes digital signal processors configured to calculate one or more features from each of one or more channels of the electrical brain activity signals. For instance, as described further below with respect to FIGS. 2-3, signal analyzer 118 may compute the power and coherence of the electrical brain activity signals with respect to theta-band activity. Stimulation controller 116 is operable to use feature values derived by the signal analyzer 118 to establish and adjust brain stimulation parameters to improve cognitive process(es) of the patient 102. Stimulation controller 116 provides the determined parameters to stimulation unit 108 so that they may be applied in the patient's stimulation therapy.

FIG. 2 is a flowchart of an example process 200 for adapting a brain stimulation therapy to improve cognitive processes of a patient. The process 200 may be used, for example, with patients suffering from epilepsy or other neurological conditions that justified implantation of a chronic brain sensing and stimulation system to track and improve cognitive processes of the patient, while eliminating or reducing the need for behavioral-based cognitive testing. A data acquisition system, e.g., signal/data acquisition unit 120, acquires electrical brain activity signals (e.g., iEEG signals) from sensing electrodes implanted in the brain of a patient (202). In some implementations, the sensing electrodes are located in the hippocampus and ANT regions of the patient's brain such that the acquired signals characterize electrical activity in the hippocampus and the ANT. Multiple sensing electrodes can be implanted in these principal regions of the brain to separately monitor electrical activity in the respective left and right portions of the ANT and hippocampus, for example. A signal analysis system, e.g., signal analyzer 118, processes the sensed brain signals to extract features from the theta-frequency band including power and coherence features (204). A stimulation control system, e.g., stimulation controller 116, determines automatically or with the aid of a clinician, and using the theta-frequency band (e.g., 2-14 Hz) features, one or more parameters for a brain stimulation therapy that are likely to improve a cognitive process of the patient such as memory encoding, memory recall, or both (206).

For example, the stimulation control system may access a model that defines target values for the theta frequency band features, where the target values are set to improve or optimize the patient's verbal memory skills or related cognitive processes. Using closed-loop feedback, stimulation therapy parameters can be adjusted to minimize a difference between the measured theta-frequency band feature values in the patient and the target feature values. For example, the stimulation frequency may be lowered to obtain theta-band power and coherence targets that correlate with improved verbal memory performance. In some implementations, the timing of stimulation onset is made to align with the timing of an anticipated cognitive event (e.g., a free recall or other verbal memory task). For instance, acute memory performance may be improved by stimulation initiated shortly before, or within a predetermined time before, the anticipated cognitive event. The system can adjust an ongoing brain stimulation therapy or initiate a brain stimulation therapy using optimized parameters to improve a cognitive process of the patient such as memory encoding and/or recall (208).

FIG. 3 is a flowchart of an example process 300 for assessing a cognitive process of a patient using theta-band features from the hippocampus and anterior nucleus of the thalamus. The process 300 may be applied, for example, to indirectly track one or more cognitive processes of a patient (e.g., memory encoding and recall) over time based on cognitive biomarkers in electrical brain activity signals. A data acquisition system, e.g., signal/data acquisition unit 120, acquires electrical brain activity signals (e.g., iEEG signals) from sensing electrodes implanted in the brain of a patient (302). In some implementations, the sensing electrodes are located in the hippocampus and ANT regions of the patient's brain such that the acquired signals characterize electrical activity in the hippocampus and the ANT. A signal analysis system, e.g., signal analyzer 118, processes the sensed brain signals to extract features from the theta-frequency band including power and coherence features (304). The theta-band frequency features are used to determine cognitive score(s) indicative of a predicted level of cognition (e.g., a predicted memory level) of the patient (306). For instance, the theta-band frequency features, and optionally additional information, can be provided as input to a model that correlates such features and information with cognition levels. The model can be a linear mixed model, a generalized linear mixed model, or another suitable regression or machine-learning model that has been trained to predict cognition levels based on theta-band biomarkers (e.g., power and coherence) and additional information. In some implementations, the model accepts as input and generates predictions further based on an amount of time that the patient has been administered chronic deep brain stimulation, historical values for the one or more features of earlier-acquired electrical brain activity signals, a frequency of deep brain stimulation administered to the patient, an indication of the patient's sleep quality, an indication of the patient's mood, demographic information about the patient, or a combination of such data. The cognitive score can be stored in memory (e.g., in a patient's medical record), presented to the patient and/or a clinician, represented in a report, or otherwise produced as output (308).

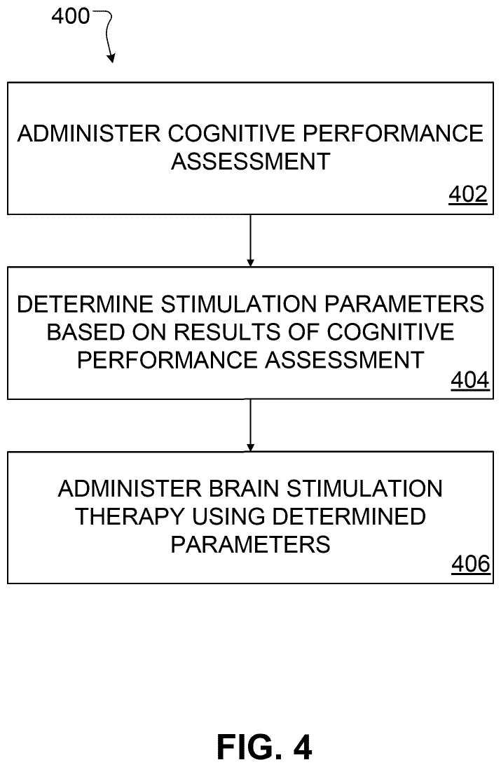

FIG. 4 is a flowchart of an example process 400 for adapting a brain stimulation therapy to improve a cognitive process of a patient. The process 400 leverages insights that can be gleaned from a patient's performance on non-invasive cognitive assessments with respect to the efficacy of a brain stimulation therapy. The patient is administered a cognitive performance assessment such as one or more free recall tasks (402). Based on the patient's performance on the cognitive performance assessments, a clinician may determine whether and how to adjust parameters of a brain stimulation therapy (404). For instance, the amount of low frequency stimulation administered to the patient may be increased with the aim of achieving chronic improvements in memory encoding, recall, or other cognitive tasks (406).

### EXAMPLE STUDY

### Abstract

Verbal memory impairment is a common symptom of temporal lobe epilepsy (“TLE”). The ability to encode and recall verbal memories can be probed with the classic free recall task. This study had three overarching hypotheses: 1) increased seizure frequency will lead to decreased verbal memory, 2) stimulation will modulate memory performance, and 3) stimulation will modulate the underlying neural correlates of memory. The study investigated these hypotheses in patients receiving chronic DBS to bilateral ANT. Recordings were obtained from individuals with drug resistant mesial TLE implanted with an investigational MEDTRONIC SUMMIT RC+S™ sensing and stimulation device. As patient seizure diaries are notoriously inaccurate, the patient's continuous local field potential (“cLFP”) was scored for seizures using a validated seizure classifier with trained epileptologist review, resulting in a reliable seizure diary. The subjects completed free recall memory tasks in their home environment with cLFP and behavioral data streamed to a handheld device and cloud repository.

Through implementation of generalized linear mixed models (GLMM), the study was able to determine that ANT stimulation modulates memory performance, but changes in the seizure rate were not predictive of changes in memory performance. Analyzing the continuous local field potential recordings from these tasks, the study found that spectral power in the theta frequency band in the left ANT and hippocampus correlated with acute memory performance and chronic therapeutic stimulation frequency received.

Not all patients are candidates for devices with chronic sensing capabilities, and this study indicates that changes in verbal memory performance can provide a suitable metric of therapy effectiveness in lieu of a reliable, objective seizure diary. But for those who do receive these and similar devices, thalamic-hippocampal spectral activities can be used to track and predict memory performance acutely and chronically, as well as predict the effect of therapeutic deep brain stimulation. Our study demonstrates chronic improvement of verbal memory with a new biomarker-based technology for remote task administration and modulation of the associated neural activities.

### Methods

Subjects. Four subjects with drug resistant epilepsy were implanted with the investigational MEDTRONIC SUMMIT RC+S™ Implantable Programmable Generator (IPG) with leads (model 3387 and 3391: 10.5 and 24 mm contact spacing, respectively) targeting bilateral anterior nucleus of the thalamus (ANT) and the hippocampus (Hc). The IPG provided continuous local field potential (LFP) time series data sampled at up to 500 Hz from four bipolar pairs selected from four out of sixteen leads. Time series data were remotely streamed to a cloud-based server accessible to clinician and researcher users.

This study was approved by the Food and Drug Administration (IDE G180224) and the Mayo Clinic Institutional Review Board. A fifth patient was consented but not implanted, and therefore is not included in this analysis. Patients received continuous or duty-cycle stimulation at either 2 Hz, 7 Hz, 100 Hz, or 145 Hz current frequency, within set ranges of pulse-width (90-200 us) and amplitude (1-6 mA). Stimulation was halted immediately before scheduled performance of memory tasks to test modulatory effects of the chronic ANT stimulation, and then turned back on upon task completion to the same settings. No testing was performed around the time changing the stimulation parameters with at least one day of continuous chronic stimulation before any testing.

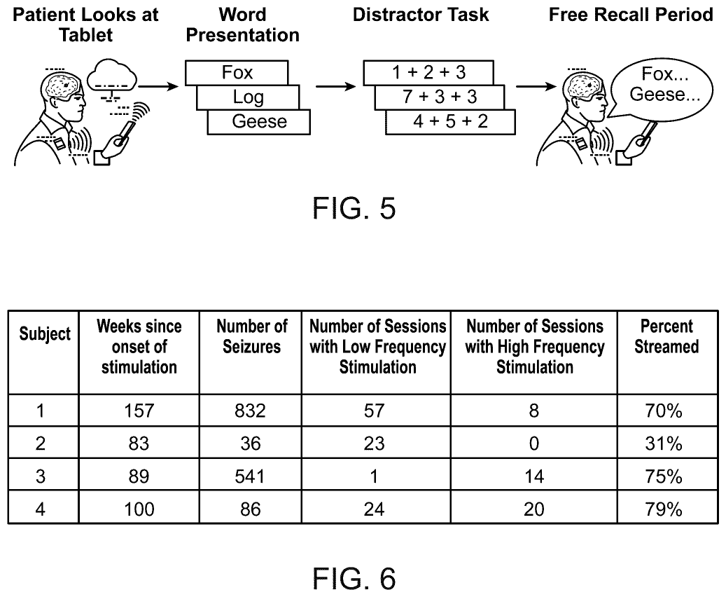

Remote Free Recall Task. Patient cognitive abilities were probed using a Free Recall Task. FIG. 5 depicts performance of an example Free Recall Task. The patients were able to complete verbal memory tasks in their home environments with LFP and behavioral data streamed to a handheld device and cloud repository. After the researcher initiated the task remotely, the participant was presented with up to 15 lists of words on a handheld tablet screen for a delayed test of free recall. Lists were composed of 12 randomly chosen nouns. Lists began with a 5 second countdown. Each word appeared on the screen for 1500 ms, with 1000 ms in between words without jitter. After a 20 s delay phase of simple addition equations answered with an onscreen keyboard, the participant was given 30 s to recall in any order as many of the words as possible recorded through a built-in microphone. Memory tasks were performed with synchronized ANT and Hc LFP data acquisition (<50 ms offset) that was artifact free due to halting stimulation for the duration of the testing. The researcher remained on the phone throughout the task. Normal subjects, or those without any cognitive dysfunction, are expected to remember 7 words per list on average.

Seizure Detection. Gold standard seizure diaries were compiled by annotating the cLFP streamed to the cloud-based server. For this study, Hc seizures were identified by a validated, high-sensitivity automated seizure classifier operating on full bandwidth Hc recordings and visually verified by a board-certified epileptologist (G. A. W. or N. M. G.).

Generalized Linear Mixed Model. This study looked at the relationship between memory scores, seizure rate from the week of the task, and whether the patient was receiving high (>100 Hz) or low (<10 Hz) frequency stimulation that week. The study used a linear mixed effect model (LMM) and a generalized linear mixed effect model (GLMM) to investigate the interactions of memory performance, seizure count, stimulation frequency, time, and patient. LMMs assume the output has a normal distribution, and GLMMs can handle data that is non-normally distributed. An LMM could be used to predict memory, but a GLMM was better suited for predicting seizures (right skewed and fitting a Poisson distribution). Both LMMs and GLMMs are particularly useful when there are mixed effects (i.e. both fixed and random effects). In this study, stimulation frequency, time, seizure count, and memory performance are all fixed effects, but patient identifier is a random effect. Since this study violates the assumption of a regular regression model that all data points are independent, patient identifier was included as a random effect to account for correlation within each patient within the regression.

To model memory, the lmer () function in R was used to create LMMs with inputs of 1) time and patient; 2) time, patient, and stimulation frequency; and 3) time, patient, stimulation frequency, and seizure count. To model seizure count, the study used the glmer () function in R to create Poisson-distributed GLMMS with inputs of 1) time and patient; 2) time, patient, and stimulation frequency; and 3) time, patient, stimulation frequency, and memory score. In all LMMs and GLMMs, time was modeled as a cubic spline, and a random intercept was used for each individual sample (patient).

The study tested whether a specific fixed effect had an effect by comparing the likelihood of a model with the predictor of interest in the model to a model without the predictor of interest included. Models were compared using a Likelihood Ratio Test. Because it was desired to compare models to test a fixed effect, the study could not use restricted maximum likelihood estimates (setting RIMI=FALSE for each model), but instead used basic maximum likelihood (ML). All likelihood tests were computed with the anova function with test=“Chisq” in R.

Power and Coherence Calculations. Hc and ANT power-in-band (PIB) measurements from the MEDTRONIC SUMMIT RC+S™ device were calculated by first downsampling the LFP signal by a factor of 5 for the theta band and a factor of 2 for the alpha band. For the beta and the broad-band gamma frequency bands, no downsampling was applied. Downsampling was applied to minimize inequalities in ratio between the sampling frequency and the upper cut-off frequency for the filter, as well as to shorten the computation time. Next, a linear trend was removed from the signal using MATLAB detrend function (MathWorks Inc.), and the obtained signal was filtered for particular frequency bands, i.e., theta (4-8 Hz), alpha (9-13 Hz), beta (13-30 Hz), and broad-band gamma (30-120 Hz). An IIR elliptic filter with passband ripple of 0.1 dB and stopband attenuation of 60 dB was used to obtain the signals in the theta, alpha and beta frequency ranges. A Kaiser window FIR filter with the same passband ripple and stopband attenuation was used to obtain the signal in the broad-band gamma frequency range. The lower and upper stopband frequencies were equal to [3.37 Hz, 10.18 Hz], [7.56 Hz, 18.81 Hz], [10.96 Hz, 44.92 Hz], [25.29 Hz, 140.41 Hz], for the four frequency bands, respectively. The filter orders were equal to 14, 12, 14, 394, respectively for each frequency band. All the filters were automatically designed by the MATLAB bandpass function (MathWorks Inc.), specific to the given frequency bands and the sampling frequency with consideration for the downsampling factor. For each filtered signal, a spectrogram was calculated using the MATLAB spectrogram function (MathWorks Inc.) which applied the short-time Fourier transform with a window size w, specified by equation 1, where fs is a sampling frequency (equal to 500 Hz in the study) and nd is a downsampling factor. Window overlap was 95%.

$$⌈ \frac{fs}{2 nd} ⌉ \qquad ( 1 )$$

Next, the spectrogram was z-scored along its rows (each 1 Hz incremental bin). Columns of the spectrogram at indices j with visible filtering artifacts, resulting from non-physiological spikes and sharp transitions, were removed from the analysis by applying a method specified by equations 2 and 3. PSD is a spectrogram of n rows by m columns, Mdn denotes a median function, and thr is a threshold value (equal to 18 in this study) for filtering out severe distortion and non-physiological spikes.

$$\text{?} = {\sum }_{i = 1}^{n} \vert \text{?} \vert \qquad ( 2 )$$

$$∀ j ∈ 1 .. m ∀ s ∈ \text{?} s > Mdn ( \text{?} ) \cdot thr \to j \qquad ( 3 )$$

$$\text{?} \text{indicates text missing or illegible when filed}$$

For each pair of filtered signals, acquired from different brain sites, a multi-taper time-frequency coherence in the form of a coherogram was calculated using the cohgramc function from the Chronux 2.11 toolbox. For the theta, alpha, and beta signals 30 tapers were used and for the broad-band gamma signal 15 tapers were used. A time-bandwidth product parameter TW was equal to 1 in every case. The movingwin parameter was equal to [1 0.01]. All the analyses were performed in MATLAB R2021b.

Spectral Statistical Analysis. The Kruskal-Wallis test was used to determine statistical significance of the differences in mean spectral power or coherence in any of the groups. In the case of the comparison between the pre/on periods, the null hypothesis was that the power/coherence means from each patient's data was not modulated by the word or equation display event. For the word display event, the “pre” and “on” periods constituted 1s before and Is after the word onset, respectively. Spearman correlation was used to determine the correlation between the memory performance (number of recalled words) and power/coherence magnitudes in distinct brain sites and task periods, across consecutive sessions. Permutation test was applied to assess statistically significant differences in memory performance (mean number of recalled words) in consecutive sessions, between the groups of different stimulation frequencies. 10,000 permutations were applied with randomized changing of labels for the stimulation session. The same approach was applied to assess statistical significance of the differences in mean power and coherence in all four brain sites and periods across consecutive sessions, between the groups of different stimulation frequencies. The significance level α in each case was equal to 0.05. All statistical tests were performed in MATLAB R2021a. The permutation test was performed using an external GPL-3.0 function permutationTest.

### Results

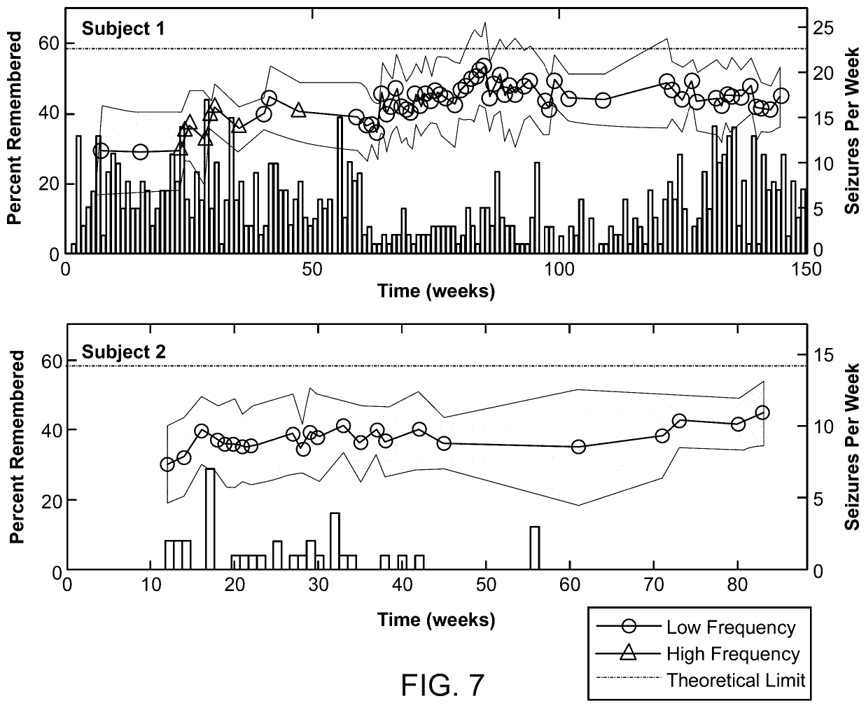

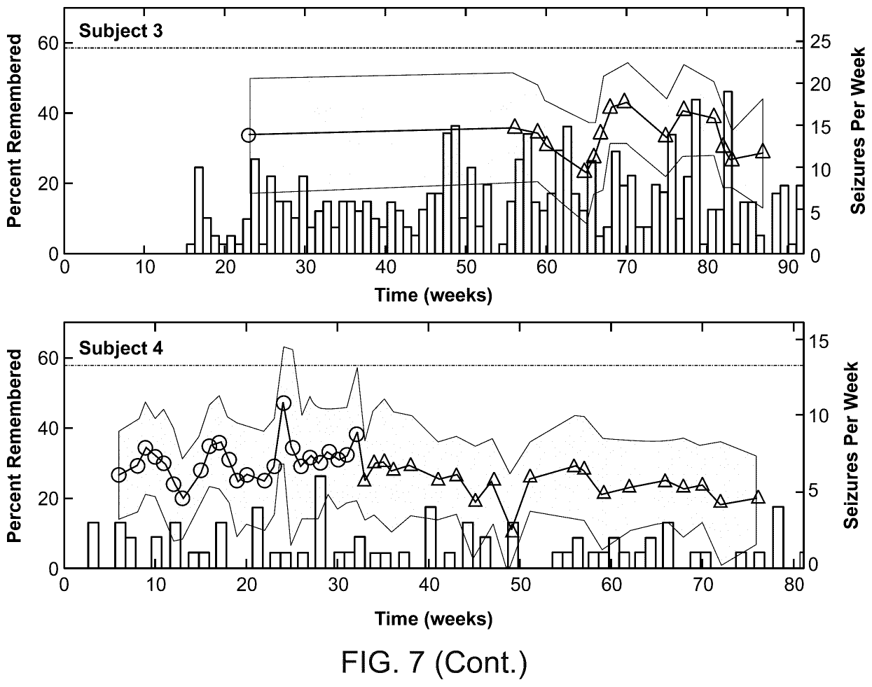

The table shown in FIG. 6 displays information on each patient's therapy length, number of seizures, number of memory task sessions, and percentage data streamed from the device. Overall, a total of 147 sessions were performed by 4 patients, with 105 tasks performed while a patient's therapeutic stimulation was set to the low frequency condition, and 42 tasks performed while it was set to the high frequency condition. Session performance compared to weekly seizure counts for all patients can be found in FIG. 7.

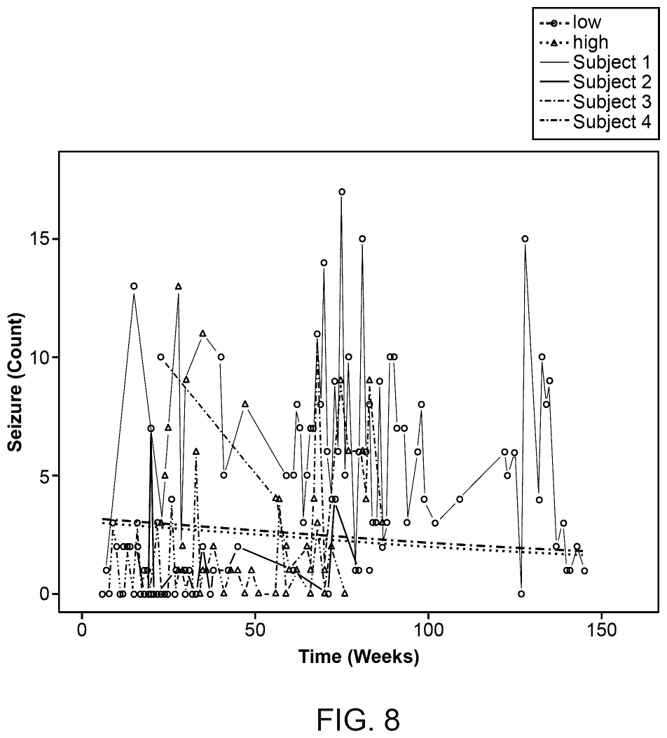

Seizure rate did not have significant relationships with stimulation or memory performance. Seizure counts were tallied for every week (as seen in FIG. 7), but only seizure counts from weeks concurrent with memory tasks were input to the GLMM. For these weeks, the study found that stimulation does not affect seizure count when accounting for time (p>0.05, p=0.6434; FIG. 8). However, it is notable that patients could decline performing tasks, so the sample may be biased toward weeks with fewer seizures. When memory score was added as an additional input to the GLMM, there was no significant change in its ability to predict seizure counts, therefore memory performance does not affect seizures when accounting for time and stimulation (p-value=0.9815).

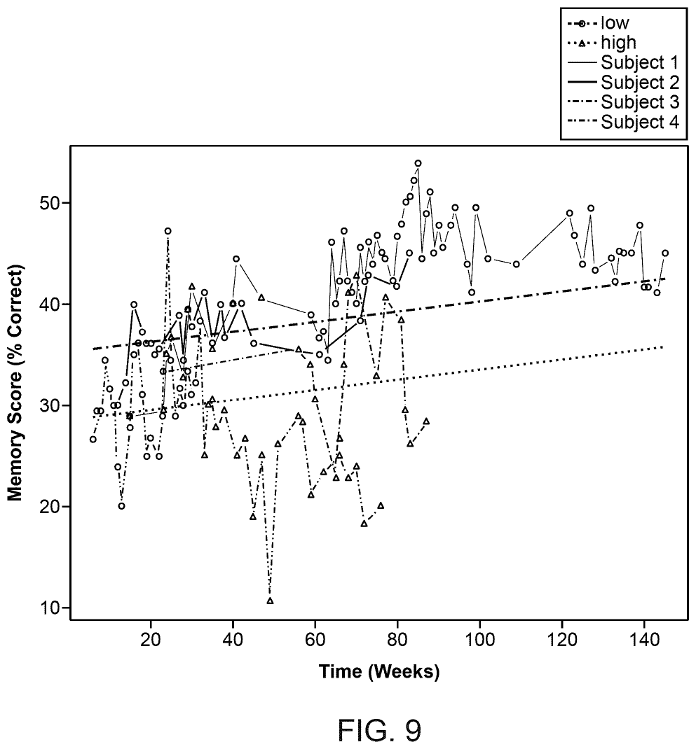

Memory Performance is Better During Low Frequency Stimulation. While the GLMM showed that weekly seizure counts do not affect memory when accounting for time and stimulation (p>0.05, p=0.9409), it did show that stimulation frequency significantly affects how patients performed on the memory task when accounting for time (p<0.001, p=2.243e-09). Memory scores were higher for weeks with low frequency stimulation than weeks with high frequency stimulation (FIG. 9).

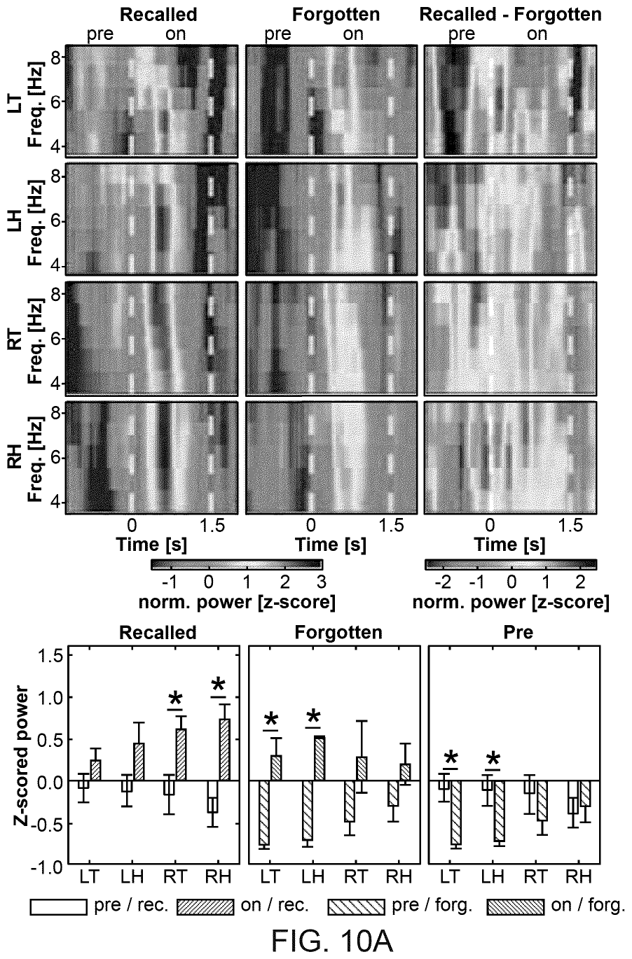

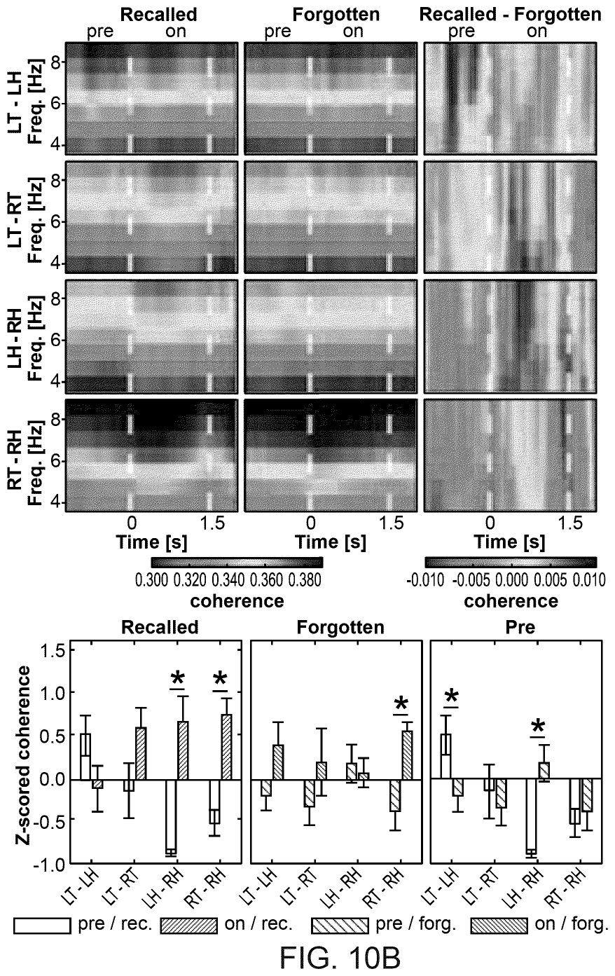

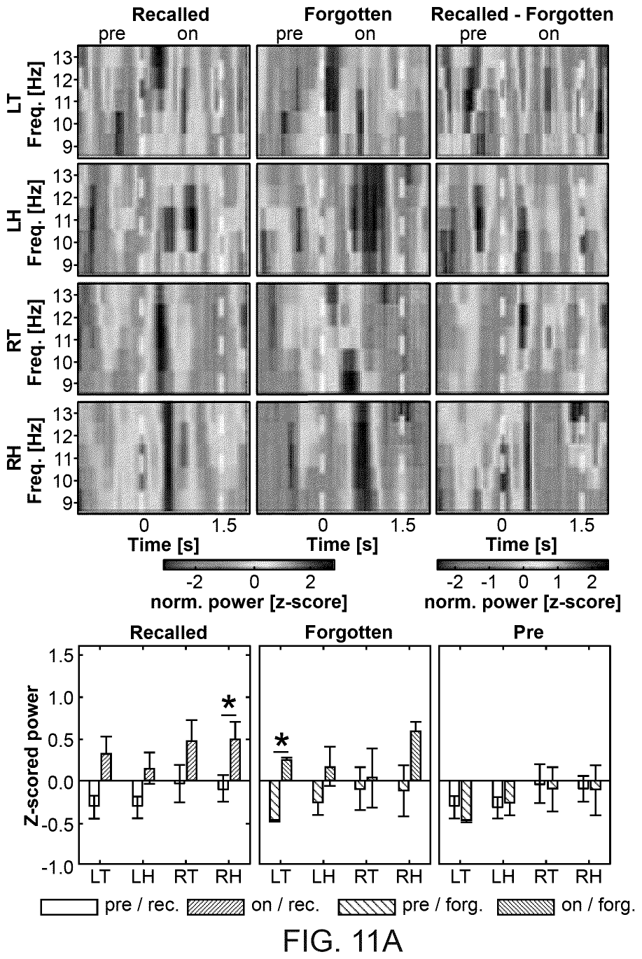

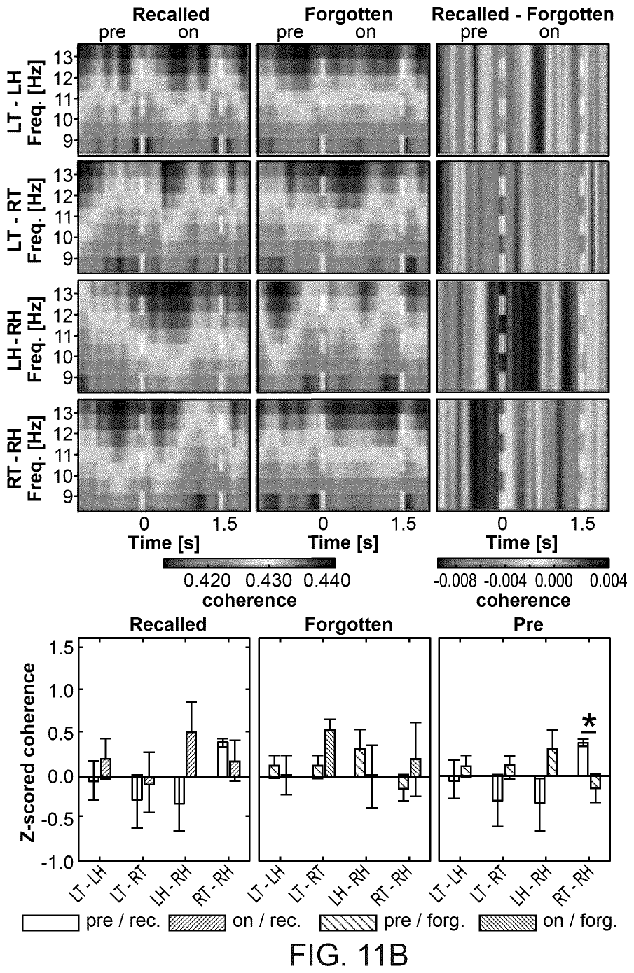

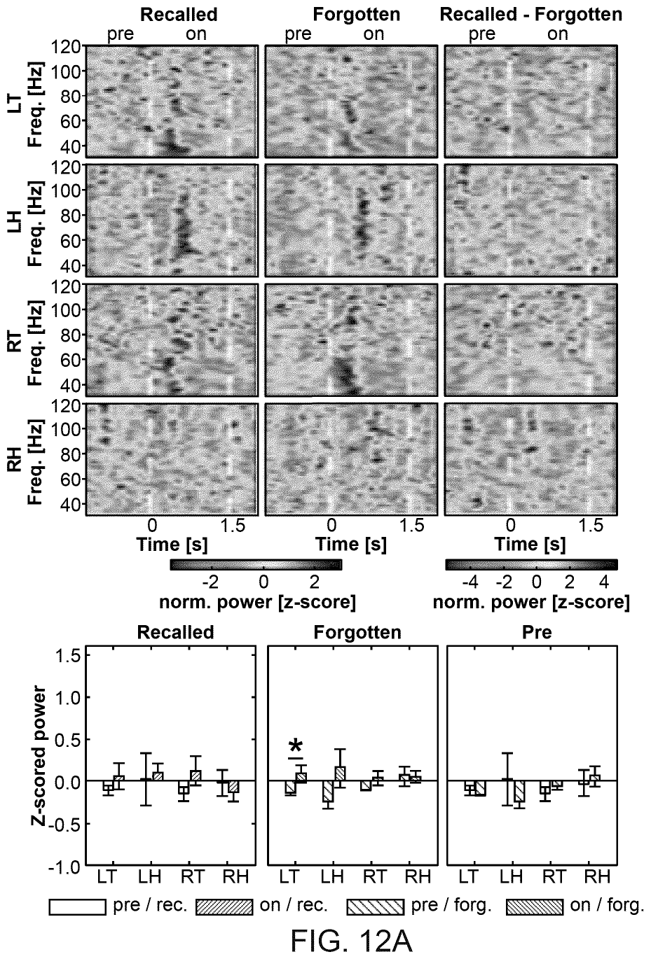

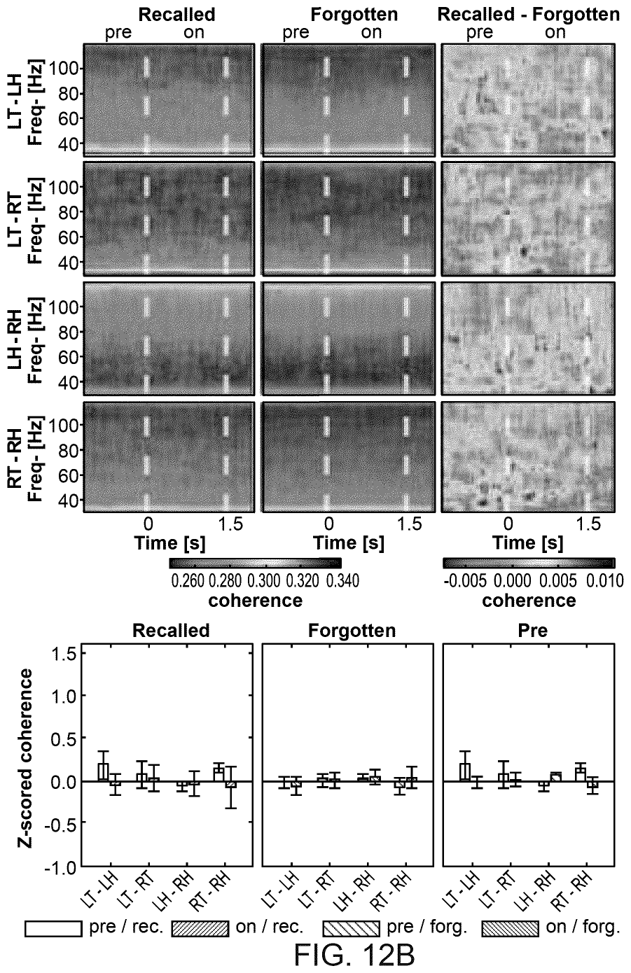

Theta Activity Predicted Subsequent Recall. The study hypothesized that theta power and coherence changes could be used to predict successful recall, i.e. show subsequent memory effect. First, the study found that task-induced changes in the theta power were different between trials with subsequently recalled and forgotten words (FIG. 10A). Theta power was significantly induced by recalled (but not forgotten) word presentation in right thalamus (RT) and right hippocampus (RH; Kruskal-Wallis, χ2=4.08, p=0.04; χ2=5.33, p=0.02, respectively). In contrast, theta power was significantly induced by the forgotten (but not the recalled) word presentation in left thalamus (LT) and left hippocampus (LH; Kruskal-Wallis, χ2=5.33, p=0.02 for both). Similar trial-specific power inductions were found in the other bands: alpha and gamma power were induced by the forgotten words in the LH (Kruskal-Wallis, χ2=5.33, p=0.02; χ2=4.08, p=0.04, respectively; FIG. 11a and FIG. 12a), and alpha power was induced by the recalled words in the RH (Kruskal-Wallis, χ2=4.08, p=0.04; FIG. 11A). Importantly, subsequent memory effect between the recalled and forgotten trials was only found in LT and LH theta power, which was significantly lower before the onset of the forgotten words (Kruskal-Wallis, LT: χ2=5.33, p=0.02; LH: χ2=4.08, p=0.04). In other words, suppressed theta power in left ANT-Hc before word onset predicted unsuccessful memory encoding.

The importance of the left ANT-Hc theta interactions for verbal memory encoding was also reflected in theta coherence (FIG. 10B). Trial-specific changes were observed between the hippocampi (LH-RH) during the recalled words (Kruskal-Wallis, χ2=5.33, p=0.02) and between RT-RH in both recalled and forgotten trial types (Kruskal-Wallis, χ2=5.33, p=0.02, in both cases). The subsequent memory effect, however, was specific to LT-LH, showing greater theta coherence immediately before presentation of the recalled compared to the forgotten words (Kruskal-Wallis, χ2=4.08, p=0.04). A reverse memory effect was found between both hippocampi (LH-RH), i.e., theta coherence was significantly lower before presentation of recalled words than forgotten (Kruskal-Wallis, χ2=5.33, p=0.02). All in all, only an increase in LT-LH theta coherence and a decrease in the LH-RH theta coherence predicted successful memory recall.

The study showed that theta power and coherence show consistent differences between successful and failed memory encoding, especially in the period immediately preceding stimulus presentation. The pattern of task-induced theta power (FIG. 10A) and coherence (FIG. 10B) was overlapping.

Chronic Memory Performance is Reflected by Thalamic-Hippocampal Theta Activity. Having established that the theta activities within and between ANT and Hc predict successful recall of verbal memory acutely, the study investigated whether they would predict general memory performance chronically across multiple sessions. It was previously found that bilateral ANT stimulation with low frequency current had a positive effect on the task performance compared to high frequency stimulation. The study first confirmed the positive effect in two patients with the longest track of recordings. The study found 43-46% improvement in the mean rate of words recalled between sessions performed during the high frequency (145 Hz) and the low frequency (2 Hz) stimulation modes (FIG. 33a; Subject 1: from 0.358 to 0.450, Subject 4: from 0.251 to 0.308).

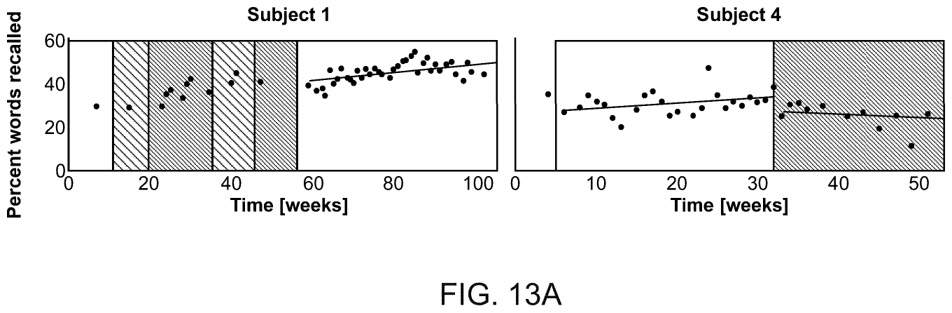

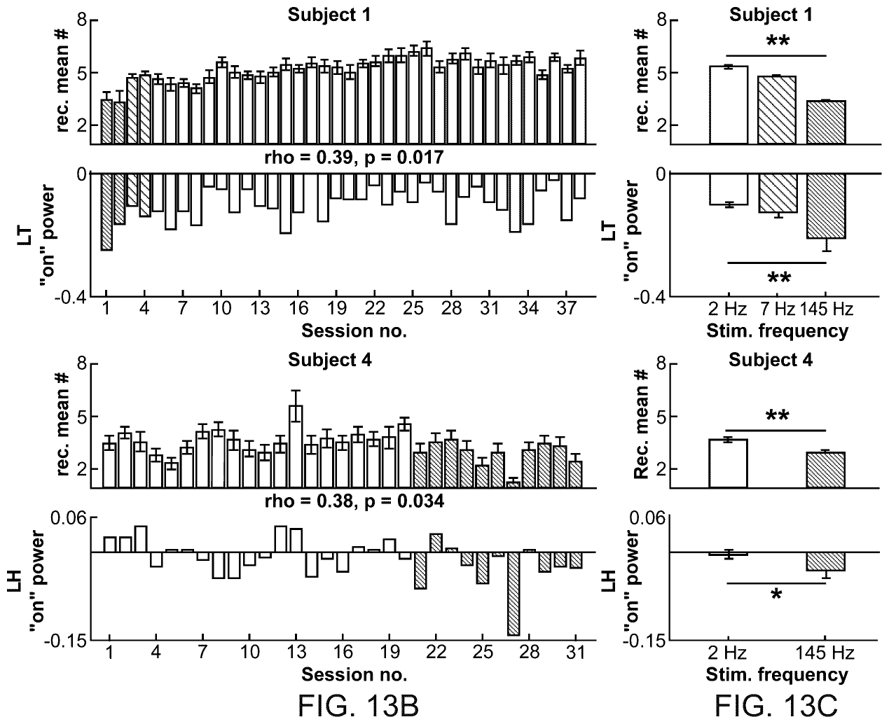

This behavioral improvement in task performance was correlated across time with the theta neural activities induced during word encoding (FIG. 13B)—similar to the acute trial-level memory effects but on the global level of sessions. There was a significant correlation of mean session performance with the LT “pre-onset” theta power in patient Subject 1 (Spearman, r=0.39, p=0.017;FIG. 33b), with the LH “pre” and “onset” theta power in patient Subject 4 (Spearman, r=0.45, p=0.011, and r=0.38, p=0.034, respectively; pre: FIG. 13B), and with “pre-onset” RT theta power in patient Subject 4 (Spearman, r=0.43, p=0.016). The other two patients had insufficient data for this longitudinal correlation with the two stimulation modes. In all these cases, higher power of the theta activities during memory encoding indicated good performance in the memory task.

Finally, the modulated theta activities reflected the effect of ANT stimulation on memory performance. Patient performance gradually improved with the low-frequency mode of stimulation (Pearson; Subject 1: r=0.56, p=0.0002; Subject 2: r=0.63, p=0.0039). In general, patients recalled more words when in the low-frequency stimulation mode (permutation test; p=0.002 for Subject 1, p=0.004 for Subject 4; FIG. 13c). Task performance during this stimulation mode revealed significantly higher theta power in LT of Subject 1 (permutation test, p=0.008), and in LH of Subject 4 (permutation test, p=0.04; FIG. 33c) compared to the high-frequency stimulation mode. This chronic modulatory effect of ANT stimulation carried over from outside of memory performance, since it was halted during the tasks.

### Discussion

Disentangling Seizures, Memory, and Stimulation. This study densely tracked memory function and seizures in people with epilepsy receiving ANT DBS. Simply calculating correlation of seizure count and memory performance provides only limited insight into the causal relationship between the two variables in time. Using a GLMM, the study was able to model the contribution of recent seizure rates and memory scores but found that seizure rate and memory score did not have significant effects on one another. However, the study did find memory scores were affected by stimulation frequency. The study notably found that in subjects living in their natural environments and receiving ANT DBS, memory performance at lower frequency stimulation was better than that during higher frequency stimulation. This may suggest that, during higher frequency stimulation, there is increased disruption of Papez circuit with associated impaired memory performance. While a verbal memory score may not stand in place of a gold standard seizure diary, the study showed that chronic verbal memory testing in clinic or remotely could provide important information to evaluate disease progress beyond seizures and to assess the effectiveness of treatments on comorbidities of epilepsy. A noninvasive metric has the potential to be particularly useful given most patients do not have a full-time, deep brain sensor.

Biomarkers for Memory and How They are Modulated by Stimulation. This study also provides a demonstration of continuous chronic deep-brain recordings from the ANT and the Hc during regular memory tasks performed remotely from real-life, home environments.

The results of this study show that the anterior thalamic-hippocampal activities of the Papez circuit are involved in verbal memory processing. Here, the study demonstrated that theta activity in this circuit is involved with verbal memory processing. Firstly, theta power and coherence were induced by presentation of words for encoding. Secondly, these induced theta activities were different during successful memory trials with subsequently recalled words, showing memory effects selectively in the left anterior thalamus and between both hippocampi. The memory effects were anatomically lateralized, pointing to the typically language-dominant left hemisphere. Finally, the same theta activities induced at the onset of words for memory encoding reflected accuracy of task performance longitudinally across multiple sessions. In other words, greater theta activity prior to word onset was associated with better verbal memory performance. Even though the activities were not the same and limited to a single anatomical site in every patient, they provide electrophysiological biomarkers of memory performance within a consistent range of frequencies in a distinct anatomical circuit. Theta activities at distinct frequencies and at particular sites of the Papez circuit may play unique roles in spatial and non-spatial memory functions, e.g. with greater involvement of the right or the left hemisphere, respectively.

The study found most of the memory-related theta activities occurred immediately before word presentation for encoding. Magnitude of both theta power and theta coherence between LT-LH was predictive of successful memory formation during attentive anticipation for the words to be presented on the screen. This suggests an underlying process related to attention and preparation for stimulus encoding—critical for memorization and subsequent recall. This preparatory activity is observed at all levels of neural activities starting from single neuron firing, as recently shown in the human hippocampus and termed “attention to encoding,” all the way to EEG theta oscillations in the thalamic nuclei. The latter showed that theta power recorded immediately before image presentation in the right anterior and mediodorsal thalamic nuclei predicted subsequent recall. In this study, the memory effect was lateralized to the left thalamus given that verbal stimuli were used instead of images. The study found theta activity was also enhanced during the delay phase of the task when the remembered items must be sustained against distractor math equations, which also points to an attention-related function (i.e. working memory). Both these neural activities and the pattern of anatomical connectivity to the anterior prefrontal cortical areas are congruent with a higher-order executive function of the anterior thalamic nuclei related to memory and attention. A hotspot of the theta activities in the left anterior prefrontal cortex in the same task was recently identified, confirming the highest magnitude of the memory effect immediately before word presentation in the cortical area directly connected to the anterior thalamic nuclei.

A strong predictive memory signal that starts even before the actual stimulus encoding presents an ideal biomarker for therapeutic interventions targeting cognition using DBS. Such biomarkers not only predict the possible outcomes of subsequent memory encoding but can provide feedback triggers for precise timing of brain stimulation in a closed-loop of LFP sensing and modulation. In the study presented here, up to 46% improvement was observed in the same verbal memory task with continuous DBS in the anterior nucleus of the thalamus. These results offer a potentially more effective strategy and/or new anatomical targets for therapeutic interventions for memory and cognitive deficits. In contrast to the widespread dispersed cortical networks, the thalamic nuclei connected to these networks are compact and condensed in confined volumes of neural tissue, making them suitable for local interventions like DBS to effectively modulate larger thalamocortical networks.

This study found evidence for chronic enhancement of verbal memory on the scale of a year, which was reflected in biomarker neural activities. The theta activities induced during memory encoding were found to correlate session-by-session with longitudinal task performance. The study was able to repeatedly test verbal memory on a weekly basis with concurrent LFP recordings in home environments. As a result, the anterior thalamic-hippocampal theta biomarkers of memory processing were demonstrated on a long timescale of regularly repeated tests. This new chronic memory biomarker predicted the effect of therapeutic stimulation and general session performance. Hence, it can be used to quantify and track memory and cognitive processes across time, which may be more objective, robust, and time-efficient than behavioral testing. It can also optimize the DBS therapy in an adaptive way over time to determine the parameters or targets of brain stimulation.

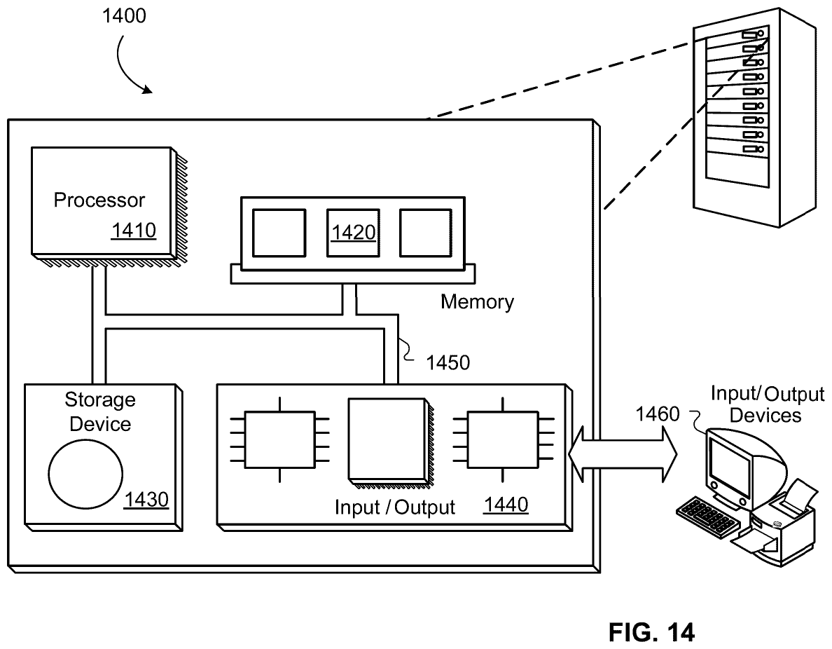

FIG. 14 shows an example computer system 1400 that includes a processor 1400, a memory 1420, a storage device 1430 and an input/output device 1440. Each of the components 1410, 1420, 1430 and 1440 can be interconnected, for example, by a system bus 1450. The processor 1410 is capable of processing instructions for execution within the system 1400. In some implementations, the processor 1410 is a single-threaded processor, a multi-threaded processor, or another type of processor. The processor 1410 is capable of processing instructions stored in the memory 1420 or on the storage device 1430. The memory 1420 and the storage device 1430 can store information within the system 1400.

The input/output device 1440 provides input/output operations for the system 1400. In some implementations, the input/output device 1440 can include one or more of a network interface device, e.g., an Ethernet card, a serial communication device, e.g., an RS-232 port, and/or a wireless interface device, e.g., an 802.11 card, a 3G wireless modem, a 4G wireless modem, a 14G wireless modem, etc. In some implementations, the input/output device can include driver devices configured to receive input data and send output data to other input/output devices, e.g., keyboard, printer and display devices 1460. In some implementations, mobile computing devices, mobile communication devices, and other devices can be used.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method of adjusting a brain stimulation therapy to affect a cognitive process of a patient, the method comprising:
acquiring, with a data acquisition system, electrical brain activity signals sensed by electrodes in a brain of the patient, the electrical brain activity signals comprising at least one signal indicative of electrical activity in a hippocampal region of the brain and at least one signal indicative of electrical activity in an anterior nucleus of the thalamus (“ANT”) region of the brain;
processing, with a signal analysis system, the electrical brain activity signals to determine values for one or more features of the electrical brain activity signals;
determining, with a stimulation control system and based on the values for the one or more features of the electrical brain activity signals, one or more parameters for the brain stimulation therapy related to the cognitive process of the patient; and
applying the brain stimulation therapy to the patient in accordance with the one or more parameters.

2. The method of claim 1, wherein:
the electrical brain activity signals comprise a first signal indicative of electrical activity in a right portion of the ANT region and a second signal indicative of electrical activity in a right portion of the hippocampal region; and
the one or more features of the electrical brain activity signals comprise at least one of a power or a coherence of theta-band electrical activity in the right portion of the ANT region and the right portion of the hippocampal region.

3. The method of claim 2, wherein the cognitive process comprises memory encoding.

4. The method of claim 1, wherein:
the electrical brain activity signals comprise a first signal indicative of electrical activity in a left portion of the ANT region and a second signal indicative of electrical activity in a left portion of the hippocampal region; and
the one or more features of the electrical brain activity signals comprise at least one of a power or a coherence of theta-band electrical activity in the left portion of the ANT region and the left portion of the hippocampal region.

5. The method of claim 4, wherein the cognitive process comprises memory recall.

6. The method of claim 1, wherein the one or more parameters for the brain stimulation therapy are determined using a model that relates at least one electrical brain activity signal feature to at least one brain stimulation therapy parameter determined to affect the cognitive process.

7. The method of claim 6, wherein the model defines target values for the one or more features of the electrical brain activity signals, and determining the one or more parameters for the brain stimulation therapy comprises comparing the values for the one or more features of the electrical brain activity signals determined from the electrical brain activity signals to the target values for the one or more features.

8. The method of claim 1, wherein the one or more parameters for the brain stimulation therapy comprise at least one of a stimulation frequency, a stimulation start time, a stimulation end time, or a selection of one or more stimulation sites in the brain of the patient.

9. The method of claim 1, wherein the brain stimulation therapy comprises a deep brain stimulation therapy (“DBS”).

10. The method of claim 1, wherein the patient is diagnosed with epilepsy and the brain stimulation therapy is administered at least in part to treat symptoms of epilepsy.

11. The method of claim 1, comprising:
identifying at least one of a type or a time of an anticipated cognitive process; and
determining the one or more parameters for the brain stimulation therapy based on at least one of the type or the time of the anticipated cognitive process.

12. The method of claim 11, comprising identifying the type of the anticipated cognitive process and determining the one or more parameters for the brain stimulation therapy based on the type of the anticipated cognitive process, wherein the type of the anticipated cognitive process is selected from a group comprising memory encoding and memory recall.

13. The method of claim 1, wherein the electrical brain activity signals comprise local field potentials (“LFPs”) sensed by the electrodes in the brain of the patient.

14. A method of assessing a cognitive process of a patient, comprising:
acquiring, with a data acquisition system, electrical brain activity signals sensed by electrodes in a brain of the patient, the electrical brain activity signals comprising at least one signal indicative of electrical activity in a hippocampal region of the brain and at least one signal indicative of electrical activity in an anterior nucleus of the thalamus (“ANT”) region of the brain;
processing, with a signal analysis system, the electrical brain activity signals to determine values for one or more features of the electrical brain activity signals;
determining, based on the values for the one or more features of the one or more electrical brain activity signals, a cognitive score indicative of a predicted level of cognition of the patient with respect to the cognitive process; and
providing an output based on the cognitive score.

15. The method of claim 14, wherein:
the electrical brain activity signals comprise a first signal indicative of electrical activity in a right portion of the ANT region and a second signal indicative of electrical activity in a right portion of the hippocampal region; and
the one or more features of the electrical brain activity signals comprise at least one of a power or a coherence of theta-band electrical activity in the right portion of the ANT region and the right portion of the hippocampal region.

16. The method of claim 15, wherein the cognitive process comprises memory encoding.

17. The method of claim 14, wherein:
the electrical brain activity signals comprise a first signal indicative of electrical activity in a left portion of the ANT region and a second signal indicative of electrical activity in a left portion of the hippocampal region; and
the one or more features of the electrical brain activity signals comprise at least one of a power or a coherence of theta-band electrical activity in the left portion of the ANT region and the left portion of the hippocampal region.

18. The method of claim 17, wherein the cognitive process comprises memory recall.

19. The method of claim 14, wherein providing the output comprises recording data indicative of the cognitive score in a memory device, presenting data indicative of the cognitive score on an electronic display, or transmitting data indicative of the cognitive score over a communications network.

20. A method of adjusting a brain stimulation therapy, comprising:
obtaining results of one or more non-invasive cognitive performance assessments administered to the patient, wherein the results are indicative of at least one of a memory encoding ability or a memory recall ability of the patient;
based on the results of the one or more non-invasive cognitive performance assessments, determining one or more parameters for the brain stimulation therapy; and
applying the brain stimulation therapy to the patient in accordance with the one or more parameters.
